(19) United States
(12) Patent Application Publication
(10) Pub. No.: US 2021/0100462 A1
(43) Pub. Date: Apr. 8, 2021

(54) PRESSURE BASED BLOOD VESSEL ASSESSMENT SYSTEMS AND METHODS

(71) Applicant: Opsens Inc., Quebec City (CA)

(72) Inventors: Claude Belleville, Quebec City (CA); André Lachance, Levis (CA); Étienne Boutin, Quebec (CA); Magalie Cantin, Lac Beauport (CA); Etienne Côté, Levis (CA)

(21) Appl. No.: 17/044,643

(22) PCT Filed: Apr. 2, 2019
(86) PCT No.: PCT/US2019/025430

§ 371 Date: Oct. 1, 2020

Related U.S. Application Data
(60) Provisional application No. 62/652,249, filed on Apr. 3, 2018.
(60) Provisional application No. 62/695,640, filed on Jul. 9, 2018.

(51) Int. Cl. A61B 5/0215; A61B 5/024; A61B 5/00

(52) U.S. Cl. CPC A61B 5/02154; A61B 5/02158; A61B 2562/0247; A61B 5/7282; A61B 2562/0233; A61B 5/024

(57) ABSTRACT

A system for assessing a vascular condition includes a pressure sensing catheter and a pressure guidewire. Heartbeats of the patient can be detected while the pressure sensing catheter and the pressure guidewire are positioned at the proximal position and at a distal position respectively. A diastolic pressure ratio zone (dPR zone) is located within a heartbeat from analysis of a signal from at least one of the pressure sensing catheter and the pressure guidewire. The dPR value can be obtained by calculating an average of several ratios of Pa to Pd taken over time within the heartbeat. A multi-beat metric (dPRc) is calculated that includes the dPR value and that also includes a high frequency sample whole heartbeat pressure ratio.

### BACKGROUND OF THE INVENTION

### Field of the Invention

This application is directed to systems and methods for determining whether and how to treat a patient based on blood pressure measurements.

### Description of the Related Art

Fractional flow reserve (FFR) is a known technique for determining whether to treat a vascular occlusion with balloon angioplasty and/or a stent. FFR is a test that is performed under hyperemia. In this technique, blood pressure is measured within the coronary vasculature distal to and proximal of the occlusion. Traditionally, a ratio of these pressures has been calculated and compared with a threshold value below which balloon angioplasty and/or stenting was indicted and above which no such treatment was to be performed.

A more recent trend has been to calculate a ratio of pressures based on data obtained at the same locations in the vasculature relative to the occlusion but based only on pressures obtained during the diastolic portion of the heartbeat cycle without hyperemia.

### SUMMARY OF THE INVENTION

Improved apparatuses and methods for determining when and how to treat coronary occlusions are needed. Such methods would advantageously be able to include data from more than just the diastolic segment and would be able to consider data from form one heartbeat cycle or more than one heartbeat cycles. Sampling from multiple heartbeat cycles and/or from multiple segments of one or multiple heartbeat cycles can provide more information about the condition of blood flow through the heart. Sampling from multiple heartbeat cycles and/or from multiple segments of one or multiple heartbeat cycles can enable clinicians to analyze cardiovascular condition during resting heartbeat cycle. Better clinical decisions flow from more comprehensive and more refined data.

Methods are provided for evaluating patients. A metric referred to herein as dPRc can be calculated. The metric uses an aortic or proximal pressure curve, referred to as a Pa curve, and a distal pressure curve, referred to as a Pd curve. The proximal pressure curve can be provided by a guide catheter pressure sensor, a pressure guidewire or another device capable of sensing pressure in the aorta. The distal pressure curve can be provided by a pressure guidewire or other device capable of sensing pressure distal to a vascular occlusion. dPRc can be a multibeat metric that incorporates data sampling from a segment of one or more adjacent beats and from one or more adjacent whole beats.

In one technique heartbeats are detected. The beats can be detected from a continuous Pa value. The beats can be detected by Pd values. The beats can be detected from both Pa values and Pd values.

In one technique, the dicrotic notch and the end of diastole (EoD) positions are recognized from the pressure data. These positions can be or can be used to define a segment of a heartbeat used to calculate a heartbeat segment metric, referred to herein as dPR. The segment from which dPR is calculate is sometimes referred to as the dPR zone. A dPR value can be calculated for each heartbeat of a series of heartbeats detected.

A whole beat metric can be calculated. The whole beat metric includes data from both systolic and diastolic parts of the heartbeat. The whole beat metric can include a pulse transmission coefficient, referred to herein as a PTC(B) value. The PTC(B) value can be calculated for each heartbeat of a series of heartbeats detected.

In some cases, a median value of PTC(B) (referred to below as PTC(B)med) is calculated over multiple heartbeats that are consecutive in time. The PTC(B)med value reduces or even in some cases minimizes the impact of signal instabilities and artefacts. A new PTC(B)med value can be calculated for each heartbeat successive. The number of consecutive heartbeats used to calculate PTC(B)med can depend on the type of analysis being performed as discussed further below.

A ratio of mean Pd to mean Pa is calculated at a sampling rate. The mean Pd to mean Pa ratio can be calculated over a period matching the most recent heartbeats used in calculating the PCT(B)med value. One new mean Pd to mean Pa ratio can be calculated for each pressure sample or measurement made. Pressure samples can be at any suitable sample rate, such as 125 hertz (every 8 ms).

The dPRc metric can be calculated for a time matching the duration of the most recent group of heartbeats used to calculate the PTC(B)med value. The dPRc value can be calculated and displayed rapidly, e.g. after each pressure sample, e.g., every 8 ms.

In one embodiment, a system is provided for assessing a vascular condition. The system includes a pressure sensing catheter, a pressure guidewire, and one or more hardware processors. The pressure sensing catheter is configured to be positioned at a proximal position within vasculature of a patient. The pressure guidewire is configured to be positioned at a distal position within the vasculature. The distal position is located distal to the proximal position. The one or more hardware processors is configured to detect heartbeats of the patient while the pressure sensing catheter and the pressure guidewire are positioned at the proximal and the distal positions in the vasculature respectively. The one or more hardware processors is configured to locate a diastolic pressure ratio (dPR) zone within a heartbeat from analysis of a signal from at least one of the pressure sensing catheter and the pressure guidewire. The one or more hardware processors is configured to calculate a dPR value including calculating an average of a plurality of ratios of Pa to Pd taken over time within the dPR zone. The one or more hardware processors is configured to calculate a multi-beat metric including the dPR value and a high frequency sample whole heartbeat pressure ratio. The one or more hardware processors is configured to output the multi-beat metric.

In one embodiment, a method of assessing a vascular condition is provided. A pressure sensing catheter is positioned at a proximal position, e.g., proximal to an occlusion within a coronary artery of a patient. A pressure guidewire is positioned at a distal position in the vasculature, e.g., distal to the occlusion. Heartbeats of the patient are detected while the pressure sensing catheter and the pressure guidewire are in the vasculature, including when positioned at the proximal position and at the distal position respectively, e.g., proximal and distal to the occlusion respectively. A diastolic pressure ratio (dPR) zone is located within a heartbeat from analysis of a signal from at least one of the pressure sensing catheter and the pressure guidewire. A dPR value is calculated. The calculation of the dPR value can include calculating an average of a plurality of ratios of Pa to Pd taken over time within the dPR zone. A multi-beat metric is calculated that includes the dPR value and that also includes a high frequency sample whole heartbeat pressure ratio. The multi-beat metric can be displayed for a user.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1 is a schematic diagram showing blood vessels with a cut-out portion in which a pressure guidewire is inserted and, spaced proximally therefrom, a guide catheter located proximally of the cut-out portion, e.g., in an aorta of a patient;

FIG. 2 is a schematic diagram of an occlusion analysis system including a pressure guidewire and a monitor assembly capable of processing vascular pressure data in connection with a vessel occlusion analysis;

FIG. 3 is a graphical representation of pressure signals over time including identification of a diastolic pressure ratio zone (dPR zone) for calculating a metric during a segment or a portion of a heartbeat cycle;

FIG. 4 is a graphical representation similar to that of FIG. 3 in connection with which a whole heartbeat cycle metric is described;

FIGS. 5-6 illustrate an analysis of multiple consecutive heartbeat cycles in calculating a multi-beat metric useful in determining whether to treat a patient;

FIG. 7 illustrates a technique for developing a stream of data for use in a static measurement inclusive of a high frequency sample pressure ratio metric as well as segment and whole heartbeat metrics over multiple consecutive heartbeats;

FIG. 8 illustrates a technique for developing a stream of data for use in a pull-back measurement inclusive of a high frequency sample pressure ratio metric as well as segment and whole heartbeat metrics over multiple consecutive heartbeats;

FIG. 8A illustrates another technique similar to that of FIG. 8 for a pull-back measurement;

FIGS. 9-13 illustrate example outputs provided on a user interface of the monitor assembly of the system of FIG. 2;

FIG. 14 is a schematic view of a blood vessel being assessed using the methodology discussed herein; and

FIG. 15 is a schematic view of a blood vessel being treated following the assessment made as illustrated in FIG. 14.

### DETAILED DESCRIPTION OF THE PREFERRED EMBODIMENT

This application is directed to systems and methods for determining whether and how to treat a patient, where data from multiple segments of heartbeat cycles and/or multiple heartbeat cycles are considered. By incorporating data indicative of both stressed and resting heart conditions, a patient condition can be more accurately assessed and improved outcomes can result.

### I. Overview of Pressure Wire Systems and Their Use

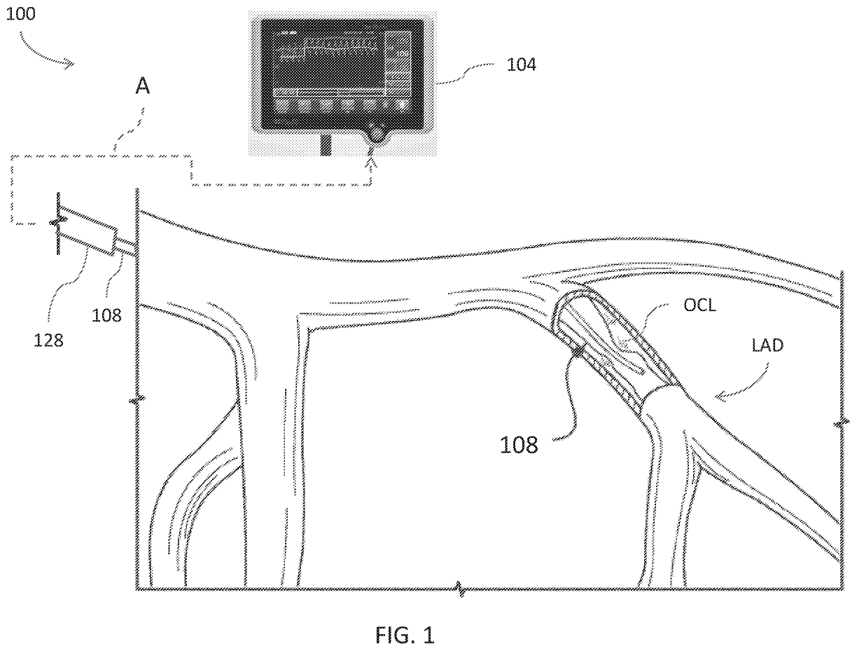

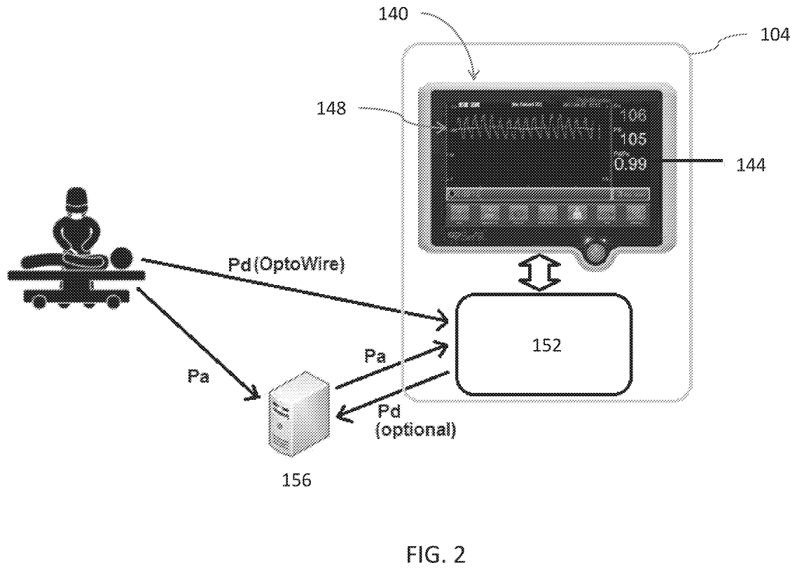

FIGS. 1 and 2 illustrate a lesion diagnostic system 100 and the use thereof in the vasculature of a patient. FIG. 1 illustrates the left side coronary vasculature with a pressure guidewire 108 disposed in a proximal portion of a left anterior descending artery (LAD). The pressure guidewire 108 is positioned in the left anterior descending artery LAD with a distal portion thereof distal to an occlusion OCL. The blood flow in the left anterior descending artery LAD is from proximal to distal, through the occlusion OCL and over the distal tip of the pressure guidewire 108. The occlusion OCL obstructs flow to at least some extent. The lesion diagnostic system 100 is configured to determine whether the extent of the obstruction is great enough to indicate that a balloon angioplasty, stent or other catheter intervention ought to be performed.

The lesion diagnostic system 100 can include a monitor assembly 104 that is configured to be coupled to the pressure guidewire 108. In one embodiment, the lesion diagnostic system 100 includes a connection (indicated by the dashed line A) that facilitates connection to and disconnection of the pressure guidewire 108 from the monitor assembly 104. The connection to and disconnection from the monitor assembly 104 is useful in allowing a clinician to use the pressure guidewire 108 initially for assessing the effect of the occlusion OCL on the flow distal thereto in the left anterior descending artery LAD (or other coronary vessel) and to use the pressure guidewire 108 at a later time for delivering a treatment device such as a balloon catheter or stent delivery system.

The connection indicated by the dashed arrow A also can couple a pressure sensing component of a guide catheter assembly 128 with the monitor assembly 104. The guide catheter assembly 128 can include a tubular catheter body used to access the vasculature. A distal tip of the guide catheter assembly 128 can be positioned proximal to the occlusion OCL such that pressure signals corresponding to the pressure proximal to the occlusion OCL, e.g., in the aorta, can be obtained. The proximal pressure is sometimes referred to herein as Pa.

The pressure guidewire 108 can take any suitable form. In one embodiment the pressure guidewire 108 includes a proximal segment that has a proximal end that is positioned outside the patient and a distal end that may be within the guide catheter assembly 128. A middle section of the pressure guidewire 108 can be configured to have the flexibility to navigate the tortuous vasculature of the left anterior descending artery LAD (or other coronary vessels) while maintaining structural integrity. A distal section can include a sensor housing and an atraumatic tip. Any sensing modality can be used. For example, an optical sensor can be configured to sense pressure when exposed to blood within left anterior descending artery LAD (or other coronary vessel). The optical sensor can be disposed within an interior space of the pressure guidewire 108 in fluid communication with an exterior of the pressure guidewire 108. The optical sensor can be selectively placed in communication with the monitor assembly 104 by a fiber optic signal line disposed between the sensor and a proximal end of the pressure guidewire 108 configured to be coupled with a fiber optic interface cable (not shown) that can include a guidewire connector to connect the pressure guidewire 108 with the rest of the system. Further details of an optical sensor based configuration of the pressure guidewire 108 can be found in US 2015/0057532, which is incorporated herein by reference in its entirety.

Where the pressure guidewire 108 is configured with an optical sensor the ability to provide a robust optical connection with the monitor assembly 104 is of interest. Any suitable connection structure or methodology can be used. One approach is described in detail in U.S. Pat. No. 9,405,078, which is incorporated by reference herein in its entirety.

FIG. 2 shows the flow of signal data more specifically. A clinician attending to the patient places the guide catheter assembly 128 in the vasculature and the pressure guidewire 108 through the guide catheter assembly 128 into the vasculature. The pressure guidewire 108 provides a signal to a processor 152 which processes the signal to determine Pd values. The processor 152 also receives Pa values from a guide catheter signal processor 156 The Pd and Pa signals are processed in the processor 152 to generate values of dPRc (as discussed further below). Those values can be displayed in a dPRc value window 144. Also, a signal trace window 148 can be provided to display traces of Pa, Pd, dPRc and/or any metrics that are combined into dPRc (as discussed below). The processor 152, the processor 152 and other processors as may be disposed in the monitor assembly 104 of elsewhere in the system 100 can be separate or combined into a single entity.

### II. Example Methodologies

### A. Metrics Combining Heartbeat Segment Analysis and Whole Heartbeat Data

An improved analysis of a patient can combine data from a segment of a heartbeat cycle with data inclusive of a whole heartbeat cycle over one or more than one consecutive heartbeat cycles.

1. Heartbeat Segment Metric—Diastolic Pressure Ratio (dPR) Calculation

In one technique, heartbeat segment data is included in a portion of a multi-beat analysis of a patient condition. A diastolic pressure ratio (dPR) calculation is an example of a heartbeat segment metric. A dPR value of a given heartbeat is determined by the mean value of a ratio of distal pressure (Pd) over proximal pressure (Pa) with a diastolic pressure ratio zone (dPR zone), as set forth in equation 1. As an example, the Pd can be measured distal to the occlusion OCL and the Pa can be measured proximal to the occlusion OCL. Pd and Pa can be measured in un-occluded vessel segments as well.

$$d P R = \frac{{\sum }_{x = x\_notch}^{x\_EoD} \frac{Pd ( x )}{Pa ( x )}}{L\_dPR} \qquad ( Eq . 1 )$$

As noted above, Pd is the pressure measured distal to the occlusion OCL and is based on pressure sensed by the pressure guidewire 108. Pa can be measured by any suitable means, such as by the guide catheter 128. Another pressure wire or other pressure sensing device could also be used to measure Pa.

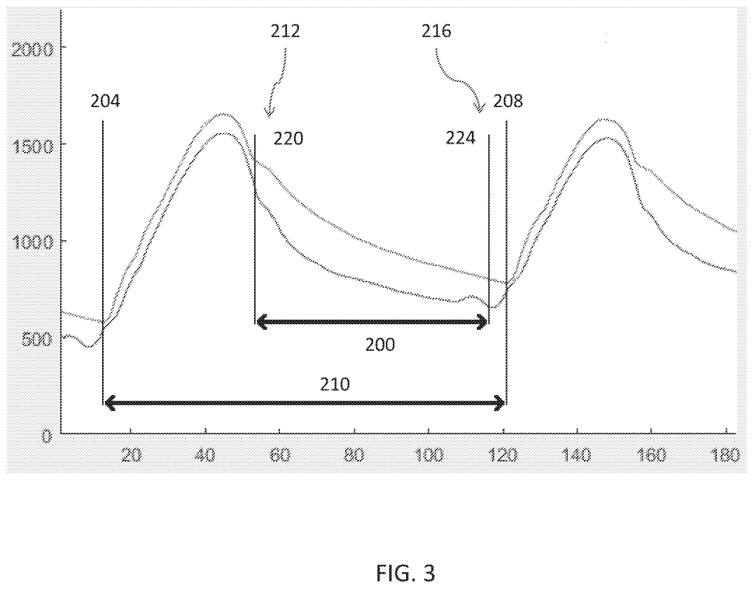

FIG. 3 shows that in one technique the dPR value is calculated based on pressure signals generated in or during a dPRzone 200. The dPRzone 200 corresponds to a segment of a heartbeat as shown in FIG. 3. The dPRzone 200 can extend from any of a number of distinct portions of the heartbeat signal or a distance therefrom. In one embodiment the dPRzone 200 is found within a first heartbeat 204. The dPRzone 200 can end prior to a second heartbeat 208. The second heartbeat 208 is immediately after the first heartbeat 204. The dPRzone 200 can be defined between the dicrotic notch 220 and the end of diastole 224 positions. FIG. 3 shows that the length of time of the dPRzone 200 is less than the time of the beat length 210. The beat length 210 can be defined as the length of time between the on-set of systole of first heartbeat 204 and the on-set of systole for the second heartbeat 208.

A new dPR value can be obtained for every detected heartbeat, e.g., for the first heartbeat 204, the second heartbeat 208, and as discussed further below, a third heartbeat 304, a fourth heartbeat 308, and a fifth heartbeat 312.

2. PTC(B) Calculation

An analysis of a patient can include whole heartbeat data as well as heartbeat segment data. For example, a pulse transfer coefficient (PTC) value can be obtained using the following method.

First a ratio of Pd to Pa is calculated. The ratio can be calculated as a ratio of the average distal pressure (Pd) during the entire beat divided by the average proximal pressure (Pa) during the entire beat. The value can be calculated using Equation 2, shown below.

$$Pd / P a = meanPdPaPeriod = \frac{{\sum }_{x = x 0 EoD}^{x 1 \_EoD} Pd ( x )}{{\sum }_{x = x 0 \_ EoD}^{x 1 \_EoD} P a ( x )} \qquad ( Eq . 2 )$$

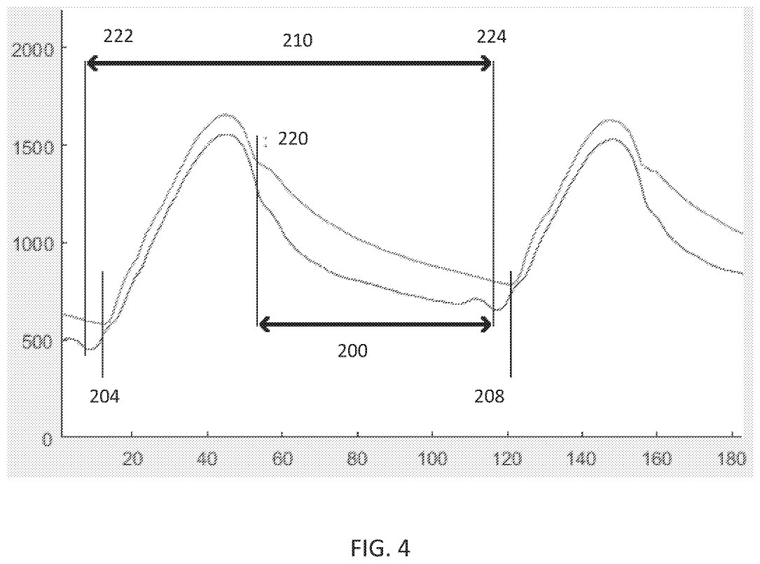

The values of Pd and Pa that are combined into the averages can be samples taken according to a sampling frequency, such as 125 hertz. FIG. 4 shows that the samples can be obtained throughout the first heartbeat 204. For example, the samples used to calculate these averages can be obtained from just after the end of diastole 222 of the heartbeat before the first heartbeat 204 (sometimes referred to herein as X0_EoD) up to the end of diastole 224 for the first heartbeat 204 (sometimes referred to herein as X1_EoD).

Any suitable approach to identify the end of diastole of the beat before the first heartbeat 204 and the end of diastole 224 of the first heartbeat 204 can be used. For example, an analysis of the pressure signals themselves from the pressure guidewire 108, the guide catheter assembly 128 or both of these devices can be used to detect the EoD. The end of diastole 222 for the prior beat can also be calculated by subtracting the beat length (however calculated) from the end of diastole 224 (however determined).

If available, an ECG signal can be used to detect these diastolic end points in other techniques.

A value of a metric including the heartbeat segment data and whole heartbeat data can thereafter be provided. In one technique a value referred to as PTC(B) can be calculated as a ratio of the heartbeat segment data to the whole heartbeat data, according to Equation 3.

$$P T C ( B ) = \frac{d P R}{Pd / P a} \qquad ( Eq . 3 )$$

This value can be calculated after the end of the first heartbeat 204 and can be calculated for subsequent heartbeats as discussed further below.

3. PTC(B)med Calculation

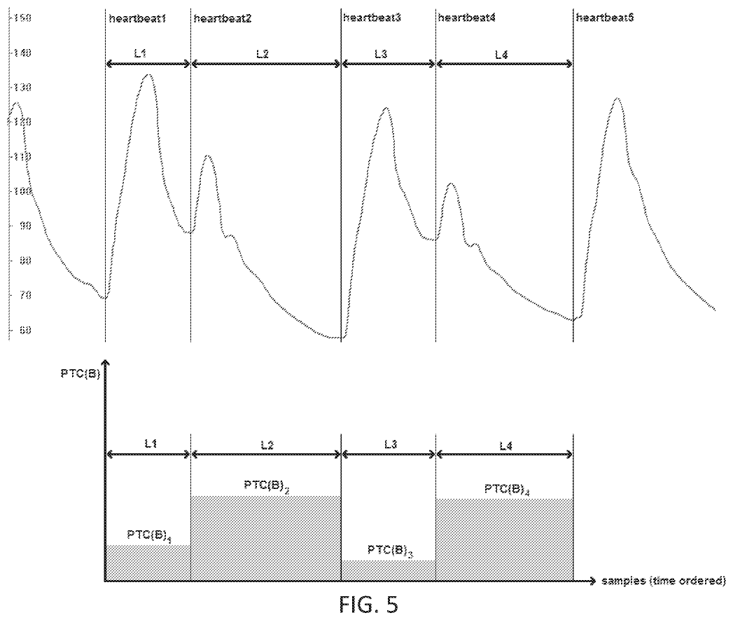

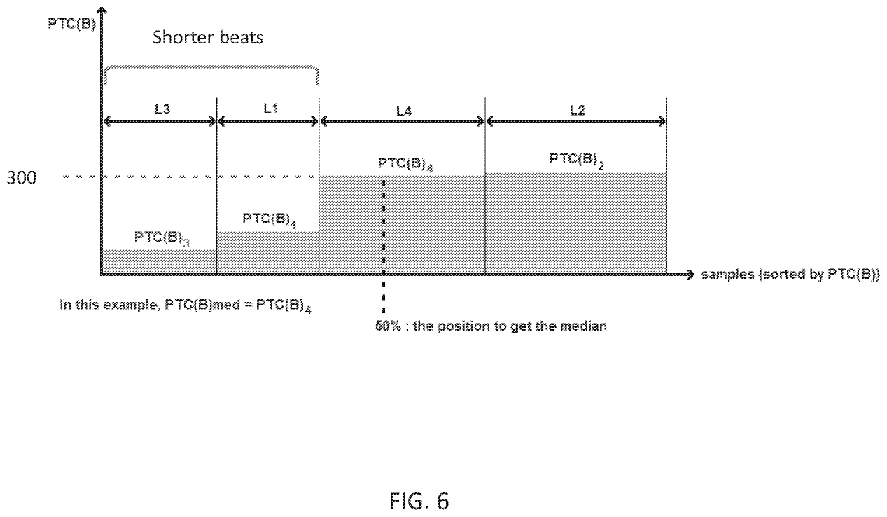

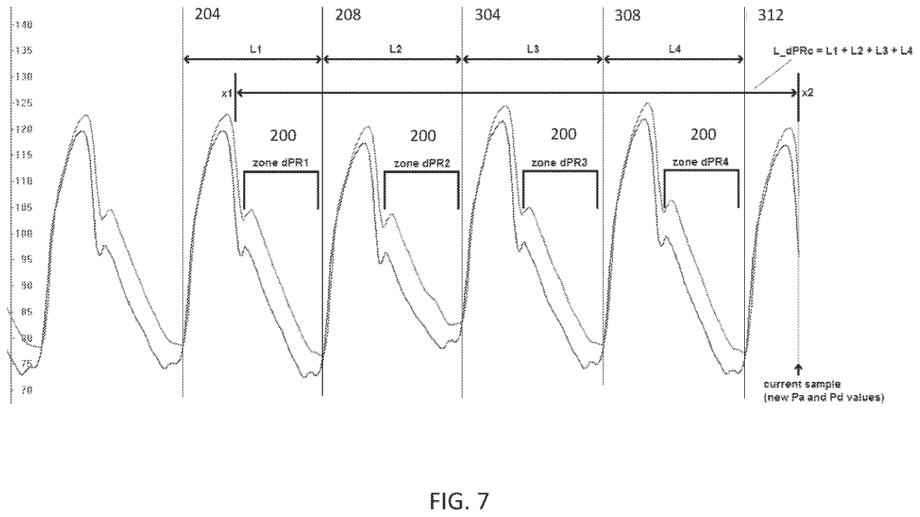

FIGS. 5-6 illustrate a further calculation of a value that considers not only heartbeat segment data and whole heartbeat data but also considers data from multiple heartbeats. As discussed further below, a multi-beat metric can include different numbers of consecutive beats depending on the test being performed.

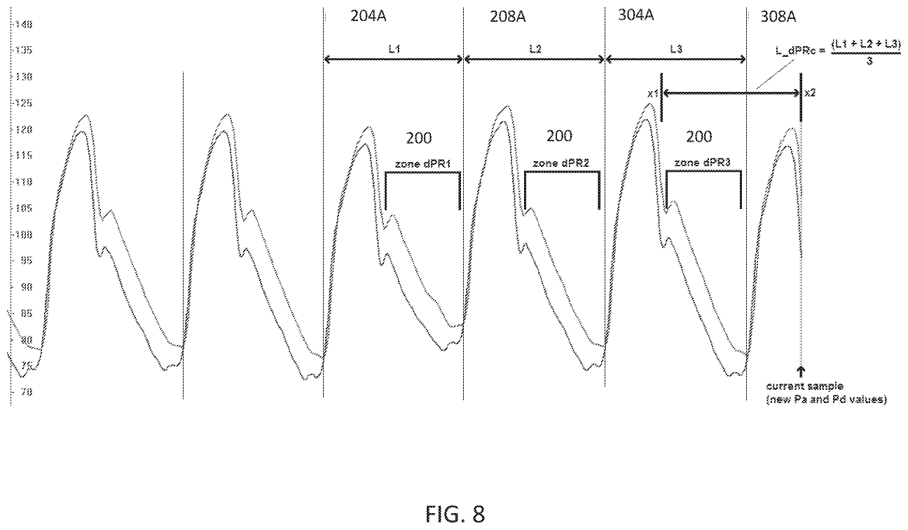

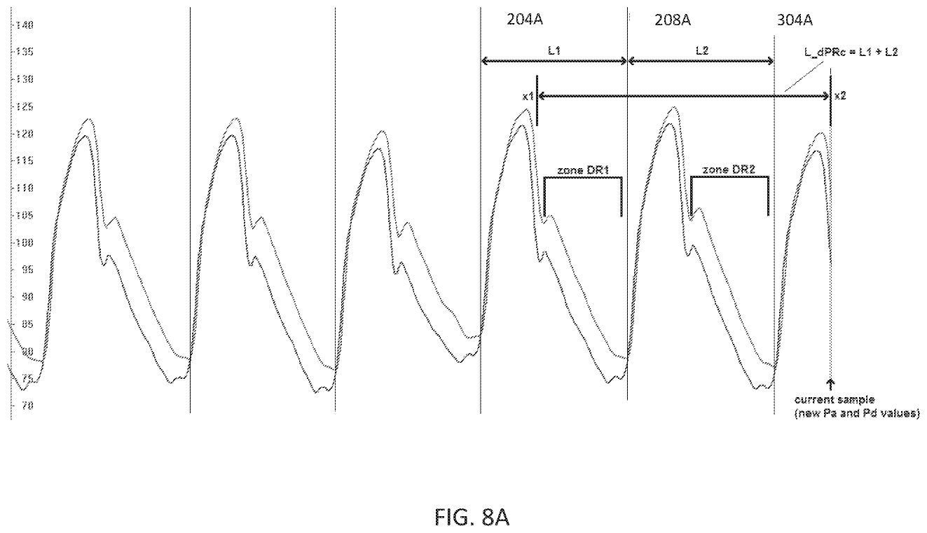

In one embodiment a multi-beat metric 300 is calculated as a value of the median of, for example, four consecutive PTC(B) values weighted based on the heartbeat length of the corresponding heartbeats. In another embodiment a multi-beat metric in connection with a pullback procedure, discussed below in connection with FIG. 8A, is calculated as a value of the median of, for example, two consecutive PTC(B) values weighted based on the heartbeat length of the corresponding heartbeats. This value is sometimes referred to herein as PTC(B)med. The purpose of this weighted median is to minimize the impact of unstable signals, such as arrhythmia or other artefacts, on metrics that include the PCT(B) value. One metric discussed below that includes PTC(B)med is a dPRc value.

One approach to calculating PTC(B)med involves the following steps. On each heartbeat period, there is a PTC(B)i value (PTC(B)1, PTC(B)2, . . . , PTC(B)N) and a period length Li (L1, L2, . . . , LN). See FIG. 5. PTC(B)med is the weighted median taken on all PTC(B)i. The weight for a PTC(B)i corresponds to the heartbeat period (Li) thereof. See FIG. 6. This way PTC(B)med is sufficiently stable even with some PTC(B) that correspond to beats that are shorter than others. On FIG. 5, PTC(B)1 and PTC(B)3 values correspond to shorter heartbeat cycles and PTC(B)2 and PTC(B)4 values correspond to beats that are longer.

In one methodology for static measurement, a new PTC(B)med is calculated for every heartbeat using all four consecutive preceding heartbeats. In another methodology for a pullback procedure, discussed below in connection with FIG. 8A, a new PTC(B)med is calculated for every heartbeat using all of two consecutive preceding heartbeats.

4. dPRc Calculation—Static Measurement

A metric combining heartbeat segment and whole heartbeat data, over multiple beats can be provided in some analyses. An example of this sort of metric is dPRc. A dPRc value is calculated as the ratio of mean Pd to mean Pa over a time period matching the duration of the four consecutive heartbeats that served to calculate the PTC(B)med, multiplied by the PTC(B)med value previously obtained. dPRc can be calculated according to Equation 4:

$$d P R c ( x ) = \frac{{\sum }_{x = xi}^{xi + L \_dPRc} Pd ( x )}{{\sum }_{x = x i}^{xi + L \_dPRc} P a ( x )} \cdot PTC ( B ) med \qquad ( Eq . 4 )$$

In this equation L_dPRc can be calculated as the sum of the length in time of the multiple beats used to calculate the current PTC(B)med value. One static measurement protocol uses four consecutive beats.

Calculating dPRc over a multiple beat (e.g., 4 beats) period provides good stability in dPRc results. It also provides a very rapid, continuous, or rapid and continuous stream of new dPRc values. This rapid stream of data is helpful in measuring conditions over time.

In case of very stable signal, dPR and dPRc results would be similar or even identical. However, in case of unstable signals, such as arrhythmia, dPRc results would be more reliable than discrete dPR values which could potentially significantly vary.

FIG. 7 illustrates how to determine end points (labeled as x1 and x2) over which the multi-beat ratio of pressure averages is calculated. x2 is the position of the current sample and x1 is obtained by subtracting L_dPRc from x2. Where L_dPRc is the sum of heartbeat periods for the beats used in calculating PTC(B)med. In the illustrated case, L_dPRc=L1+L2+L3+L4. Because a delay is required to detect any heartbeat (analyzing many samples), there is always a delay between x2 and the last heartbeat detected.

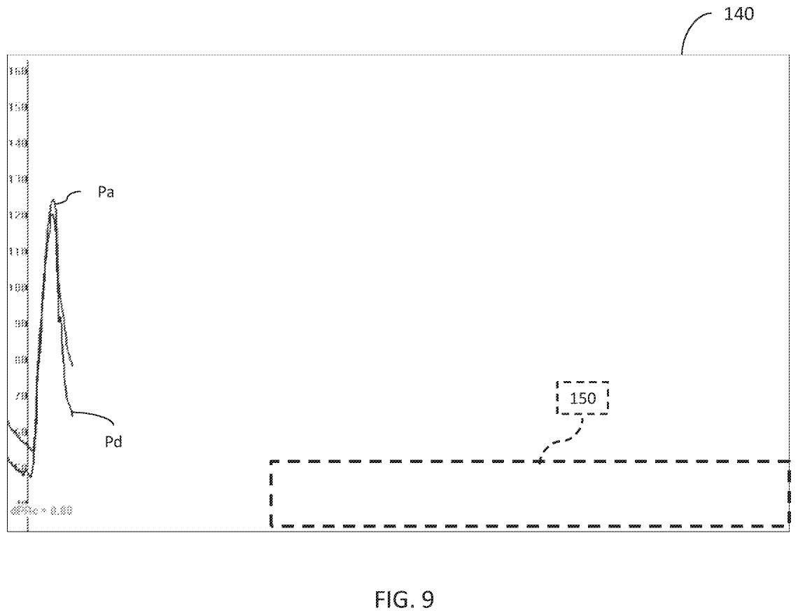

FIGS. 9-13 illustrate how the foregoing could be displayed on the signal trace window 148 or in another part of the user interface 140 of the monitor 104. In each figure, the Pa and Pd traces are displayed and labeled. At any given point in time there will generally be a lower value for Pd than for Pa in the case where the occlusion OCL is impeding flow downstream thereof. The blue vertical lines above the trace represent the separate heartbeats. The horizontal line beneath the traces labeled “dPR” correspond to each dPR zone 200.

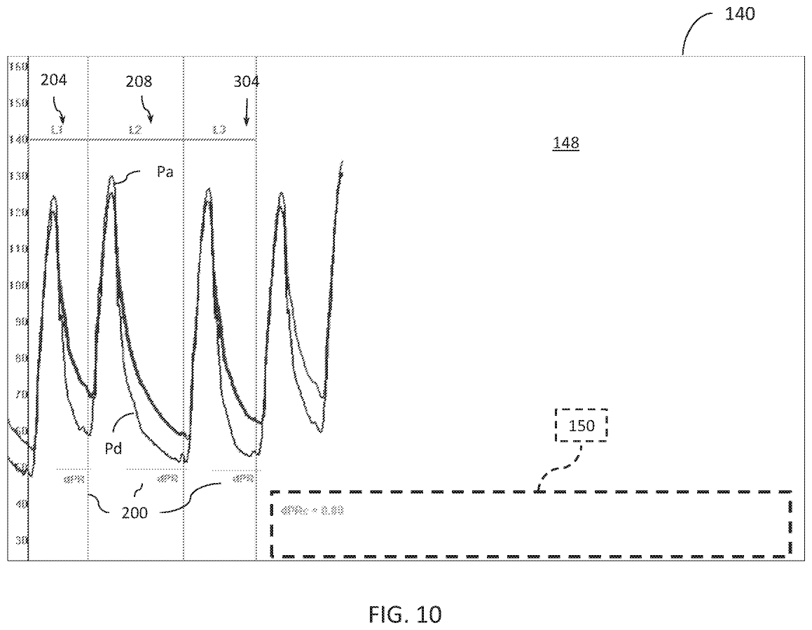

FIG. 9 shows an initial portion of an analysis of pressure data from the pressure guidewire 108 and the guide catheter assembly 128. The initial portion includes the rising pressures associated with systole and the decreasing pressures associated with the on-set and initial portions of diastole in the first heartbeat 204. FIG. 9 shows only a part of the first heartbeat 204. FIG. 10 shows the first heartbeat 204, the second heartbeat 208, and the third heartbeat 304. For each beat the dPR value can be calculated as described above in the corresponding dPRzone 200.

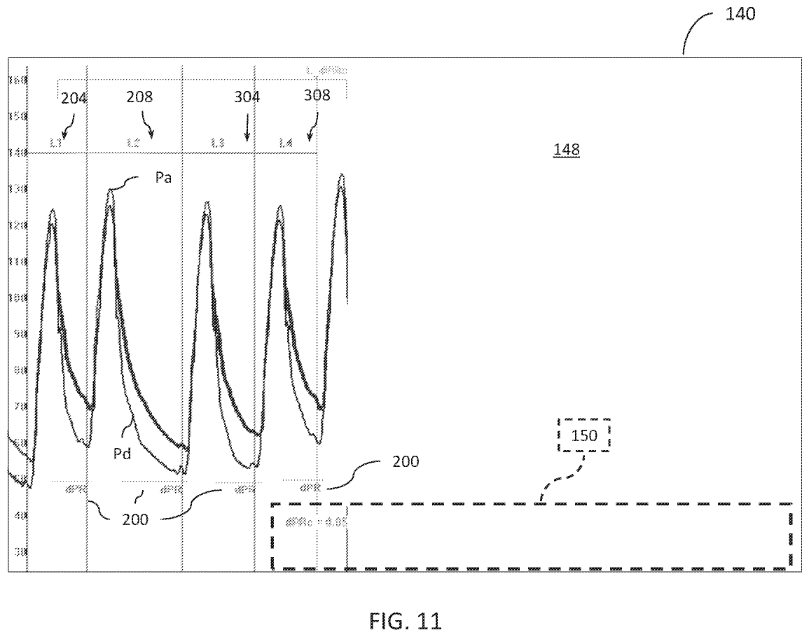

FIG. 11 shows the first, second, and third beats and the fourth heartbeat 308. After the first heartbeat 204, second heartbeat 208, third heartbeat 304, and fourth heartbeat 308 have been detected and analyzed dPRc or another multi-beat metric combining segment and whole beat data can be calculated for these four beats. The user interface 140 can be configured to include a dPRc trace window 150 to display dPRc or another multi-beat metric combining segment and whole beat data. FIG. 10 shows that prior to sufficient consecutive beats being detected a 0 value can be displayed for dPRc and no trace is presented in the dPRc trace window 150. After four (or another sufficient number of beats) have been detected and analyzed the dPRc trace window 150 can be modified to display one or both of a dPRc value and a dPRc trace as shown in FIG. 11.

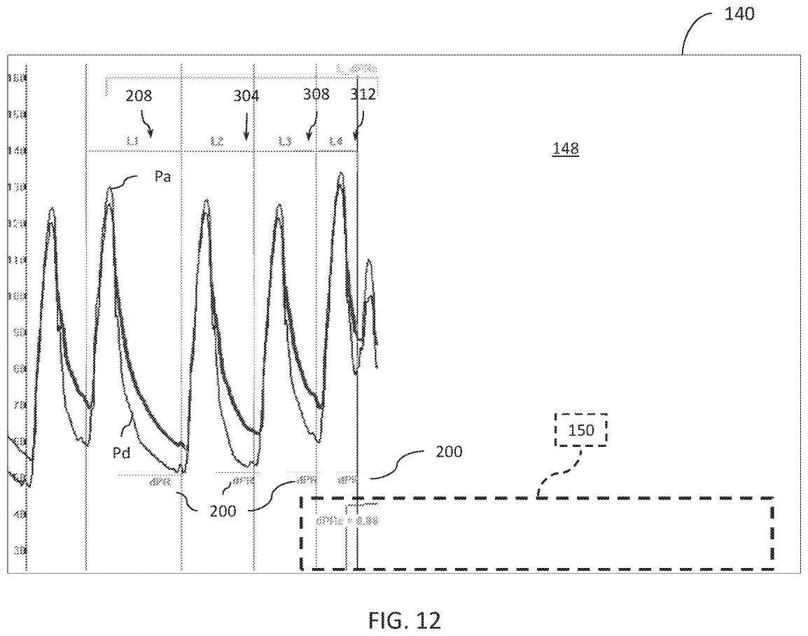

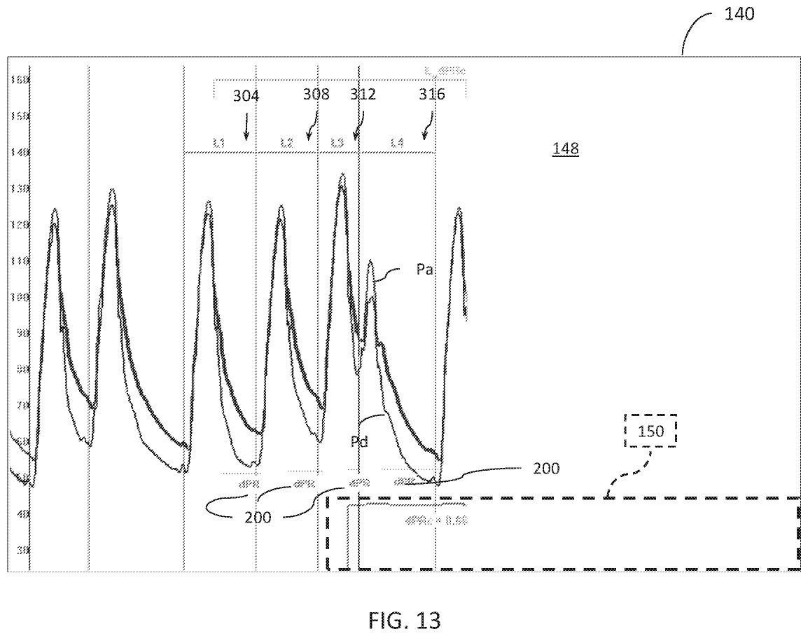

FIG. 12 shows how the user interface 140 illustrates that the analysis of dPRc is updated for fifth and subsequent consecutive beats. A new dPRc value is calculated based on the first heartbeat 204, the third heartbeat 304, the fourth heartbeat 308, and a fifth heartbeat 312. The new dPRc value is generated following the same protocol noted above, where PTC(B)median is the weighted median of the second, third, fourth and fifth beats and the pressure ratio multiplier in equation 4 is based on a new time period of L_dPRc as the sum of the beat lengths for the second heartbeat 208, third heartbeat 304, fourth heartbeat 308, and fifth heartbeat 312 (sum of L1, L2, L3, and L4). The new dPRc value and/or the dPRc trace is updated in the dPRc trace window 150 on the user interface 140. FIG. 13 shows further calculation of the dPRc metric later in time, using the third heartbeat 304, the fourth heartbeat 308, the fifth heartbeat 312, and a sixth heartbeat 316. Again, the new dPRc value and/or the dPRc trace is updated in the dPRc trace window 150 on the user interface 140.

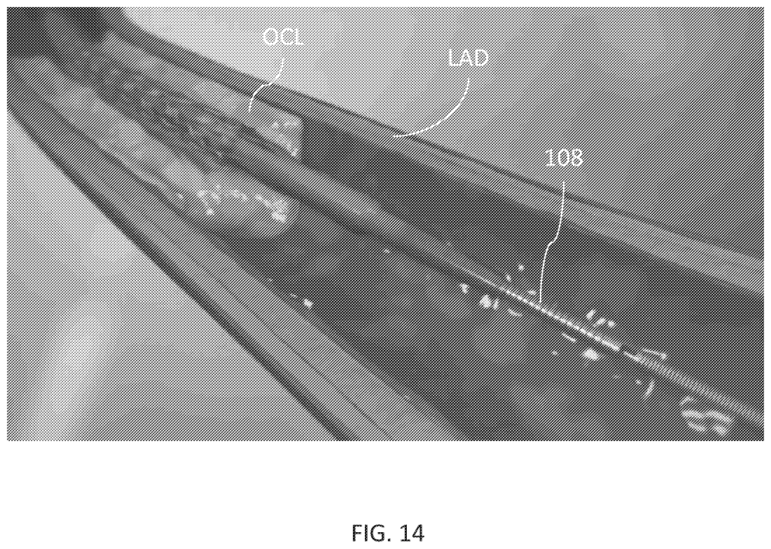

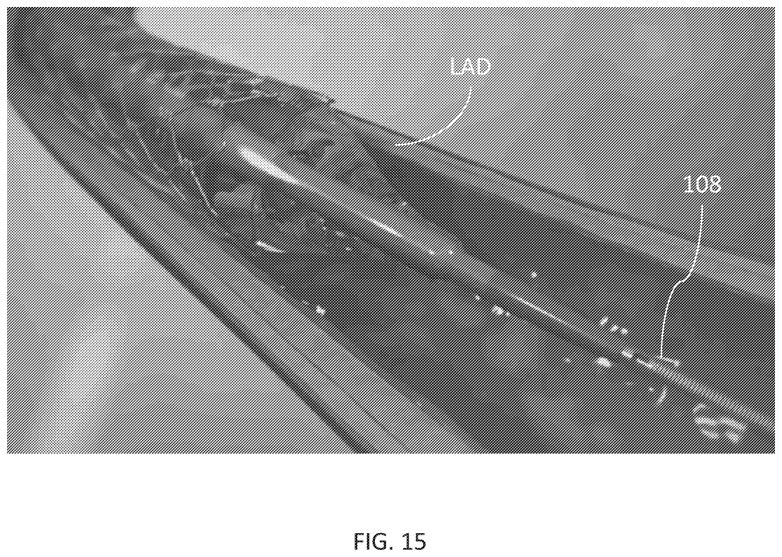

Based on the analysis, a threshold value can be established above which a patient is not treated and below which a treatment such as angioplasty or stenting is performed. As shown in FIGS. 14 and 15 both the assessment of dPRc and the treatment can be performed over the pressure guidewire 108. By updating the dPRc value over time the user can see the stability of the metric and gain confidence in next clinical steps, such as whether to treat with a balloon, a stent or other method. Also, the output in the dPRc trace window 150 can be updated as fast as the samples of Pa and Pd are taken, e.g., every 8 ms based on a sampling rate of 125 hertz. In some cases, the screen can be updated less frequently but still much faster than every second, e.g., 30 times per second. This protocol provides effectively a continuous stream of data, e.g., a stream of data updated more often than every heartbeat, updated more than once per second, updated more than twice per second, updated more five times per second, updated more than ten times per second, updated more fifty times per second, updated more than one hundred times per second.

5. dPRc Calculation—Pullback Measurement

While the foregoing has been focused largely on a static position measurement, that is one made with at least the pressure guidewire 108 held stationary, another mode involves obtaining pressure data and analyzing the data while at least the pressure guidewire 108 is moving. Generally the movement of the guidewire 108 that is provided is in the proximal direction from a distal position in the vasculature toward a proximal position adjacent to the distal end of the guide catheter assembly 128. This motion can be provided by the clinician pulling back on the pressure guidewire 108 directly manually or using a device configured to generate a controlled proximal movement.

FIG. 8 illustrates one embodiment of a pullback mode analysis. In this example, dPRc is calculated by Equation 4.

$$d P R c ( x ) = \frac{{\sum }_{x = xi}^{xi + L \_dPRc} Pd ( x )}{{\sum }_{x = x i}^{xi + L \_dPRc} P a ( x )} \cdot PTC ( B ) med \qquad ( Eq . 4 )$$

One difference, however, is PTC(B)med can be based on the most recent three beats. Also, L_dPRc is the average period of the three beats (e.g., a first best 204A, a second best 208A, and a third beat 304A) used to calculate PTC(B)med. In other words, the first term in Equation 4 is the average distal pressure over the time L_dPRc divided by the average proximal pressure over the time L_dPRc. FIG. 8 shows the window between x1 and x2 as between the time of the current pressure sample data back by the amount of L_dPRc.

FIG. 8A shows another technique for conducting an analysis in a pullback mode. This technique is similar to that if FIG. 8 except as described differently below. Here two beats (204A, 208A) are used in calculating PTC(B)med. This value is multiplied by the ratio of Pd/Pa, calculated as expressed in Equation 4. However, in this calculation L_dPRc is the sum of the period of the two beats, shown as the time between X1 and X2. This can be calculated as the time between the start of systole for the beat 204A and the time for systole for the beat 304A. The window for calculating the Pd/Pa will shift out in time for each new sample, e.g., every 8 milliseconds. The value of L_dPRc can be calculated every time a new value of PTC(B)med is calculated, e.g., after the end of each full beat. One advantage of the approach discussed in connection FIG. 8A is that is provides faster response time than an approach requiring more than two beats to present a pullback mode value. If a more stable value is desired more beats can be used, similar to the method of FIG. 8. Another advantage of the algorithm discussed in connection with FIG. 8A is that is includes an analogous calculation as is used for the static or stationary mode, but using two beats rather than four as used in the static or stationary mode.

The foregoing approaches to dPRc provides a rapid stream of data over time which provides more clarity for the pullback mode.

### B. Advantages

The foregoing discusses using an average of a plurality of ratios of Pd to Pa as part of calculating a useful blood vessel occlusion evaluation metric. The averaging of these ratios provides advantages. For example, whenever noise is present the average of the ratios is more accurate than other manners of combining multiple measurements, such as calculating a ratio of an average of multiple distal pressure measurements to an average of multiple proximal pressure measurements. This is particularly true whenever the Pa exhibits large pressure excursion caused by pressure tube movement or other similar sources of noise.

The dPRc method including the calculation of PTC(B)med allows reliable dPR calculation without the need for analyzing and removing any data associated with heartbeats that may actually be irregular in some way. This method thus can be carried out without any need to determine a priori any and all criteria that would justify removing or discarding data associated with irregular heartbeats.

In pull back technique, a faster stream of data is available, allowing rapid response of the dPRc measurement and hence, enhanced spatial resolution.

### Terminology

As used herein, the relative terms “proximal” and “distal” shall be defined from the perspective of the user of the system. Thus, proximal refers to the direction toward the user of the system and distal refers to the direction away from the user of the system.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The terms “approximately,” “about,” “generally,” and “substantially” as used herein represent an amount close to the stated amount that still performs a desired function or achieves a desired result. For example, the terms “approximately,” “about,” “generally,” and “substantially” may refer to an amount that is within less than 10% of the stated amount, as the context may dictate.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about four” includes “four”

Any methods disclosed herein need not be performed in the order recited. The methods disclosed herein include certain actions taken by a practitioner; however, they can also include any third-party instruction of those actions, either expressly or by implication. For example, actions such as “distally moving a locking element” include “instructing distal movement of the locking element.”

Although certain embodiments and examples have been described herein, it will be understood by those skilled in the art that many aspects of the humeral assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. A system for assessing a vascular condition, comprising:
a pressure sensing catheter configured to be positioned at a proximal position within vasculature of a patient;
a pressure guidewire configured to be positioned at a distal position within the vasculature, the distal position being located distal to the proximal position;
one or more hardware processors configured:
detect heartbeats of the patient while the pressure sensing catheter and the pressure guidewire are positioned at the proximal and the distal positions in the vasculature respectively;
locate a diastolic pressure ratio (dPR) zone within a heartbeat from analysis of a signal from at least one of the pressure sensing catheter and the pressure guidewire;
calculate a dPR value including calculating an average of a plurality of ratios of Pa to Pd taken over time within the dPR zone;
calculate a multi-beat metric including the dPR value and a high frequency sample whole heartbeat pressure ratio; and
output the multi-beat metric.

2. The system of claim 1, wherein processor is configured to calculate the whole heartbeat pressure ratio with samples from systolic and diastolic periods of at least two consecutive heartbeats.

3. The system of claim 1 wherein processor is configured to calculate the whole heartbeat metric from data from a first window including multiple consecutive heartbeats and wherein the high frequency sample whole heartbeat pressure ratio is calculated from data from a second window having a length corresponding to that of the first window, the second window partially overlapping but not coterminous with the first window.

4. The system of claim 1 wherein processor is configured to calculate the whole heartbeat metric is calculated from data from a first window including multiple consecutive heartbeats and wherein the high frequency sample whole heartbeat pressure is calculated from data from a second window having a length equal to an average period of the heartbeats within the first window, the second window overlapping and end portion of the first window.

5. The system of claim 1 wherein the processor is configured to calculate the multi-beat metric according to the formula
$$dPR c ( x ) = \frac{{\sum }_{x = xi}^{xi + {L}_{dPRc}} Pd ( x )}{{\sum }_{x = x i}^{xi + {L}_{dPRc}} P a ( x )} \cdot PTC ( B ) med$$

6. A method of assessing a vascular condition, comprising:
positioning a pressure sensing catheter in vasculature of a patient at a proximal position within vasculature of a patient;
positioning a pressure guidewire at a distal position, the distal position being located distal to the proximal position;
detecting heartbeats of the patient while the pressure sensing catheter and the pressure guidewire are positioned proximal and distal in the vasculature respectively;
locating a diastolic pressure ratio (dPR) zone within a heartbeat from analysis of a signal from at least one of the pressure sensing catheter and the pressure guidewire;
calculating a dPR value including calculating an average of a plurality of ratios of Pa to Pd taken over time within the dPR zone;
calculating a multi-beat metric including the dPR value and a high frequency sample whole heartbeat pressure ratio; and
displaying for a user the multi-beat metric.

7. The method of claim 6, wherein detecting heartbeats comprises analyzing a continuous signal from at least one of the pressure guidewire (Pd) and the pressure sensing catheter (Pa).

8. The method of claim 6, wherein locating the dPR zone comprises identifying a dicrotic notch position and an end of diastole position from analysis of the signal from at least one of the pressure sensing catheter and the pressure guidewire.

9. The method of claim 6, wherein the dPR value for the heartbeat is calculated as
$$d P R = \frac{{\sum }_{x = x\_notch}^{x\_EoD} \frac{Pd ( x )}{Pa ( x )}}{L\_dPR}$$

10. The method of claim 6, wherein the high frequency sample whole heartbeat metric is calculated as
$$Pd / P a = meanPdPaPeriod = \frac{{\sum }_{x = x 0 EoD}^{x 1 \_EoD} Pd ( x )}{{\sum }_{x = x 0 \_ EoD}^{x 1 \_EoD} P a ( x )}$$

11. The method of claim 6, wherein multi-beat metric includes a calculation of a median value for a plurality of consecutive heartbeats of
$$P T C ( B ) = \frac{d P R}{Pd / P a}$$

12. The method of claim 11, wherein the pressure guidewire is held stationary and the median value is based on four consecutive heartbeats.

13. The method of claim 12, wherein the multi-beat metric is calculated as
$$dPR c ( x ) = \frac{{\sum }_{x = xi}^{xi + L \_dPRc} Pd ( x )}{{\sum }_{x = x i}^{xi + L \_dPRc} P a ( x )} \cdot PTC ( B ) med$$
where L_dPRc is a time corresponding to the sum of periods of four consecutive heartbeats.

14. The method of claim 11, wherein the pressure guidewire is moved proximally in a pullback mode and the median value is based on two or three consecutive heartbeats.

15. The method of claim 14, wherein the multi-beat metric is calculated as
$$dPR c ( x ) = \frac{{\sum }_{x = xi}^{xi + L \_dPRc} Pd ( x )}{{\sum }_{x = x i}^{xi + L \_dPRc} P a ( x )} \cdot PTC ( B ) med$$
where L_dPRc is a time corresponding to an average of the period of three consecutive heartbeats.

16. The method of claim 14, wherein the multi-beat metric is calculated as
$$dPR c ( x ) = \frac{{\sum }_{x = xi}^{xi + L \_dPRc} Pd ( x )}{{\sum }_{x = x i}^{xi + L \_dPRc} P a ( x )} \cdot PTC ( B ) med$$
where L_dPRc is a time corresponding to the sum of the period of two consecutive heartbeats.

17. The method of claim 6, wherein the whole heartbeat pressure ratio is calculated based on a sampling frequency of 125 Hz.

18. The method of claim 1, wherein the multi-beat metric is calculated according to the formula
$$dPR c ( x ) = \frac{{\sum }_{x = xi}^{xi + L \_dPRc} Pd ( x )}{{\sum }_{x = x i}^{xi + L \_dPRc} P a ( x )} \cdot PTC ( B ) med$$

19. The method of claim 17, further comprising recalculating the multi-beat metric a plurality of times within a heartbeat cycle and displaying the re-calculated whole heartbeat metric a plurality of times within a heartbeat cycle.

20. The method of claim 6, wherein the whole heartbeat pressure ratio includes samples from systolic and diastolic periods of two consecutive heartbeats.

21. The method of claim 6, wherein the whole heartbeat metric includes samples from systolic and diastolic periods of at least three consecutive heartbeats.

22. The method of claim 6, wherein the whole heartbeat metric includes samples from systolic and diastolic periods of at least four consecutive heartbeats.

23. The method of claim 6 wherein the whole heartbeat metric is calculated from data from a first window including multiple consecutive heartbeats and wherein the high frequency sample whole heartbeat pressure ratio is calculated from data from a second window having a length corresponding to that of the first window, the second window partially overlapping but not coterminous with the first window.

24. The method of claim 6 wherein calculating the whole heartbeat metric is calculated from data from a first window including multiple consecutive heartbeats and wherein the high frequency sample whole heartbeat pressure is calculated from data from a second window having a length equal to an average period of the heartbeats within the first window, the second window overlapping and end portion of the first window.
